(19) United States
(12) Patent Application Publication
(10) Pub. No.: US 2006/0292653 A1
(43) Pub. Date: Dec. 28, 2006

(54) Metal mediated fluorescence superquenching assays for detection of phosphorylation and dephosphorylation of quencher-labeled substrates

(76) Inventors: Frauke Rininsland, Santa Fe, NM (US); Casey Stankewicz, Madison, WI (US)

Correspondence Address: DLA PIPER US LLP;ATTN: PATENT GROUP, 1200 NINETEENTH STREET, NW, WASHINGTON, DC, 20036, US

(21) Appl. No.: 11/378,470

(22) Filed: Mar. 20, 2006

Related U.S. Application Data
(60) Provisional application No. 60/662,783, filed on Mar. 18, 2005.
(60) Provisional application No. 60/705,495, filed on Aug. 5, 2005.

(51) Int. Cl. C12Q 1/48; C12Q 1/42

(52) U.S. Cl. 435/15; 435/21

(57) ABSTRACT

A method includes contacting an analyte; a phosphorylatable or dephosphorylatable substrate bound to a quencher; and a sensor that includes at least one fluorescent polymer complexed to one or more metal species; to form a composition; and determining the fluorescence of the composition, if any; wherein when the substrate is phosphorylated, an association occurs between the substrate and the sensor such that fluorescent superquenching occurs between the fluorescent polymer and the quencher.

### CROSS REFERENCE TO RELATED APPLICATIONS

This application is based on, and claims priority to, U.S. Provisional Application Ser. No. 60/662,783, filed Mar. 18, 2005, and U.S. Provisional Application Serial No. 60/705,495, filed Aug. 5, 2005, the entire contents of which are hereby incorporated by reference.

### TECHNICAL FIELD

The present application relates generally to reagents, kits and assays for the detection of biological molecules and, in particular, to reagents, kits and assays for the detection of biological molecules which combine metal or metal ion binding and fluorescent polymer superquenching and which employ quencher labeled substrates.

### BACKGROUND

Phosphorylation and dephosphorylation of proteins, polynucleotides, or lipids control the regulation of cellular metabolism, growth, differentiation, and proliferation. Aberrations in enzymatic function can lead to diseases such as cancer, inflammation, and others. More than 500 kinases and phosphatases are thought to be involved in the regulation of cellular activity and are possible targets for drug therapy.

The phosphoinositide 3-kinase (PI3K) family of kinases is an important part of one of the major pathways of intracellular signal transduction. Signals mediated by the PI3 kinase enzyme influence a wide variety of cellular functions, including cell growth, differentiation and survival, glucose metabolism, and cytoskeletal organization. Many cell-surface receptors, and particularly those linked to tyrosine kinases, are known to activate PI3 kinases. Many cellular functions and events appear to be influenced by the lipid products generated by these enzymes. Different isoforms of the enzyme have specialized functions. The development of therapeutically useful, isoform-selective inhibitors with limited toxicity would be invaluable against cancer or diseases with an inflammatory or immune component.

The primary enzymatic activity of the PI3 kinases is the phosphorylation of inositol lipids at the 3-position. Different members of the PI3 kinase family generate different lipid products. To date, four 3′-phosphorylated inositol lipids have been identified in vivo of which phosphatidylinositol (3,4)-bisphosphate (PtdIns(3,4)P₂)and PtdIns(3,4,5)-trisphosphate (PtdIns(3,4,5)P₃, or PIP₃) act as second messengers. The PI3 kinases are divided into three classes (Classes I, II and III) on the basis of their structure, binding partners/adapters, mode of activation, and substrate specificity in vitro.

PI3 kinases are activated by growth factors, insulin and G-protein coupled receptors. Class I PI3K's are divided into two subclasses, IA and IB, on the basis of their mechanisms of activation. The four Class I PI3K's currently known (α, β, γ, and δ) phosphorylate the 3-hydroxyl group of the inositol ring of phosphatidylinositol (PtdIns), PtdIns(4)P, and PtdIns(4,5)P₂ to form PtdIns(3)P, PtdIns(3,4)P₂, and PtdIns(3,4,5)P₃ (also called PIP3). PIP3 binds to a variety of effector molecules including serine-threonine kinases (such as protein kinase B), tyrosine kinases (Tec family members), and regulators of G-proteins (such as GRP1). These effector molecules control cell growth and proliferation, prevent apoptosis and mediate the metabolic actions of insulin.

The known functional effects of the PI3 kinases make them possible therapeutic targets in the treatment of inflammatory, malignant, and immunological diseases, for example, cancer and diabetes. PI3 kinase inhibition might prove to be useful in benign proliferative conditions, such as the vascular smooth muscle proliferation following balloon angioplasty that can lead to re-stenosis, diabetic proliferative retinopathy, and idiopathic fibrotic diseases such as fibrosing alveolitis.

Several methods are currently used to measure PI3K activity. These include monitoring of incorporated radioactive ³²P (from ATP-γ³²P), extraction of radioactive compounds, and separation using thin layer chromatography. These and other radioactive methods have several disadvantages such as safety hazards, reagent disposal costs, and heterogeneity involving wash steps that are work intensive and time consuming. Non-radioactive methods have been developed, which include high performance liquid chromatography (HPLC), enzyme-linked immunosorbent assays (ELISA), fluorescence polarization (FP), AlphaScreen and time-resolved fluorescence resonance energy transfer (FRET). None of these are satisfactory for high-throughput screening (HTS), however. HPLC methods are expensive, time-consuming, and unsuitable for high-throughput screening applications. ELISA-based assays are cumbersome, time-consuming and heterogeneous. FP, AlphaScreen and FRET assays are indirect, competitive assays and are less sensitive than direct, homogeneous assays. They are prone to interference from fluorescent compounds in a compound library, and yield false positives and negatives that require the screener to resort to further testing of the hits from a primary screen.

Due to the lack of HTS compatible techniques for screening of inhibitors for PI3K activities, only a few PI3K inhibitors, wortmannin, LY-294002, and quercetin dihydrate, have been identified. A number of chemical modifications of wortmannin and LY294002 have been made, a few of which are significantly more potent than the parent compound. The lack of selectivity of both compounds for the isoforms of PI3K enzymes, however, together with the instability of wortmannin and the insolubility of LY294002 means that neither has promising pharmaceutical potential.

There is a need for sensitive, homogeneous, safe and reliable assays that show low interference from fluorescent library compounds, and that rapidly and accurately detect and quantify biologically relevant molecules such as enzymes and nucleic acids with high sensitivity.

### SUMMARY

One embodiment provides a method, which includes:

contacting,
an analyte;
a phosphorylatable or dephosphorylatable substrate bound to a quencher; and
a sensor that includes at least one fluorescent polymer complexed to one or more metal species;

to form a composition; and

determining the fluorescence of the composition, if any;

wherein when the substrate is phosphorylated, an association occurs between the substrate and the sensor such that fluorescent superquenching occurs between the fluorescent polymer and the quencher.

Another embodiment provides a method for detecting an inhibitor and/or activator of PI3 kinase or phosphatase activity, which includes:

contacting,
an analyte;
a phosphorylatable or dephosphorylatable substrate, specific to PI3 phosphatase or kinase, bound to a quencher; and
a sensor that includes at least one fluorescent polymer complexed to one or more metal species;

to form a composition; and

determining the fluorescence of the composition, if any;

wherein when the substrate is phosphorylated, an association occurs between the substrate and the sensor such that fluorescent superquenching occurs between the fluorescent polymer and the quencher.

Another embodiment provides an assay kit for probing an analyte, which includes:
a phosphorylatable or dephosphorylatable substrate bound to a quencher; and
a sensor that includes at least one fluorescent polymer complexed to one or more metal species;

wherein when the sensor and the substrate bound to the quencher are present in intimate admixture, and wherein when the substrate is phosphorylated, an association occurs between the substrate and the sensor such that fluorescent superquenching occurs between the fluorescent polymer and the quencher.

### DESCRIPTION OF THE DRAWINGS

FIG. 1 shows an illustration of one polymer used in metal ion mediated fluorescence superquenching assays.

FIG. 2 shows assays developed using quencher-labeled synthetic lipids as substrates. Upon phosphorylation of the substrate, the lipid associates to the sensor via the phosphate groups and quenches fluorescence.

FIG. 3 shows one schematic for a kinase enzyme activity sensor based on metal ion mediated fluorescence superquenching.

FIG. 4 shows a calibrator curve using phosphatidylinositol 3 (PtdIns).

FIG. 5 shows an enzyme concentration curve of phophoinositol kinases (PI3K). α, β, and γ using a quencher labeled phosphatidylinositol as a substrate.

FIG. 6 shows PI3K (α, β, δ) inhibition by wortmannin (A) and PI3Kα inhibition by wortmannin and LY-294002 (B).

FIG. 7 shows one schematic of a sensing platform based on polynucleotide kinase activity.

FIG. 8 shows an enzyme concentration curve for PI3Kα monitored in four different modes (A=polymer fluorescence quench mode; B=quencher re-emission mode; C=in ratiometric re-emission mode and D=in ratiometric quench mode).

FIG. 9 shows results of a 84 compound screen against PI3Kδ in ratiometric turn off mode

FIG. 10 shows results of a follow up screen for “Hits” identified in the compound screen for PI3Kδ.

Tables 1 and 2 show statistical parametric data that can be delivered with an assay according to one embodiment.

### DESCRIPTION OF THE SEVERAL EMBODIMENTS

According to one embodiment, a bioconjugate is provided which includes:

a polynucleotide or lipid comprising one or more phosphorylatable or dephosphorylatable groups; and

a quenching moiety bound or tethered to the polynucleotide or lipid, wherein the quenching moiety is capable of amplified super-quenching of a fluorescent polymer comprising a plurality of fluorescent species when associated therewith.

In one embodiment, the quenching moiety can be rhodamine. The polynucleotide or lipid can comprise one or more phosphate groups.

According to another embodiment, a method of detecting the presence and/or amount of an enzyme analyte in a sample is provided which includes:

a) contacting the sample with the bioconjugate as set forth above, wherein the polynucleotide or lipid of the bioconjugate comprises one or more groups which are phosphorylatable or dephosphorylatable by the enzyme analyte;

b) contacting the sample with a fluorescent polymer comprising a plurality of fluorescent species associated with one another such that the quenching moiety is capable of amplified superquenching of the fluorescent polymer when the quenching moiety is associated with the fluorescent polymer, wherein the fluorescent polymer further comprises one or more anionic groups and wherein at least one metal cation is associated with an anionic group of the fluorescent polymer; and

c) detecting fluorescence from the sample;

wherein the detected fluorescence indicates the presence and/or amount of analyte in the sample.

The polynucleotide or lipid can include one or more groups which are phosphorylatable by the analyte such that phosphorylation of the phosphorylatable groups of the polynucleotide or lipid results in a decrease in fluorescence. Alternatively, the polynucleotide or lipid can include one or more groups which are dephosphorylatable by the analyte such that dephosphorylation of the dephosphorylatable groups of the polynucleotide or lipid results in an increase in fluorescence.

In one embodiment, the metal cation can be Ga³⁺.

The fluorescer can be a fluorescent polymer such as a poly(p-phenylene-ethynylene) polymer comprising anionic groups (e.g., a sulfo poly p-phenyleneethynylene (PPE-Di-COOH)).

The fluorescer can be associated with the surface of a solid support (e.g., a microsphere, plastic, glass, magnetic, polystyrene, bead, textured, flat, any combination thereof, and the like). In one embodiment the surface is a polystyrene bead having a diameter ranging from 0.2-8 μm. This range includes all values and subranges therebetween including 0.2, 0.25, 0.3, 0.35, 0.4, 0.45, 0.5, 0.55, 0.6, 0.65. 0.7, 0.75, and 0.8 μm. The solid support can comprise a positively charged surface, and one or more anionic groups of the fluorescer (e.g., the fluorescent polymer) can be associated with the positively charged surface of the solid support.

The fluorescer can be added to the sample after incubation and before detecting fluorescence. Alternatively, the fluorescer can be added to the sample before incubation or during incubation and detecting fluorescence can include detecting fluorescence during incubation.

According to another embodiment, a kit for detecting the presence and/or amount of a kinase or phosphatase enzyme analyte in a sample is provided which includes:

a first component comprising a bioconjugate as set forth above; and

a second component comprising a fluorescent polymer, the fluorescent polymer comprising a plurality of fluorescent species associated with one another such that the quenching moiety of the bioconjugate is capable of amplified superquenching of the fluorescent polymer when the quenching moiety is associated with the fluorescent polymer, wherein the fluorescent polymer optionally further comprises one or more anionic groups and wherein at least one metal cation is associated with an anionic group of the fluorescent polymer when the anionic groups are present.

One embodiment includes a more sensitive, homogenous assay for inhibitors and/or activators of PI3K activity. Another embodiment includes compounds that are potent inhibitors of the PI3K enzymes, which can be revealed by the assay.

One embodiment identifies inhibitors and/or activators of the phosphoinositide 3-kinases, alpha, beta, and delta (PI3K α, β, γ, and δ) activities by ratiometric monitoring of metal ion mediated superquenching alone, or in combination with enhanced emission, using the fluorescent polymer/metal sensor and the quencher-labeled substrate.

Another embodiment identifies inhibitors and activators of the phosphoinositide 3-kinases, alpha, beta, and delta (PI3K α, β, γ, and δ) activities and developing pharmaceutical compositions that are useful in the treatment of diseases responsive to the inhibition or activation of the PI3K enzymes.

One embodiment provides a new assay based on fluorescent polymer superquenching that is sensitive, homogenous and utilizes a ratiometric read-out method that reduces or eliminates interference from fluorescent and colored compounds. Using this assay, a screen of 84 compounds was done against PI3K 6 enzyme, and eight novel inhibitors of the enzyme were identified by the present inventors as truly strong inhibitors. These “hit” compounds may be suitably formulated into pharmaceutical compositions for treatment of diseases responsive to inhibitors of PI3K enzymes.

One embodiment utilizes a lipid which is labeled with a fluorescent quencher dye as substrate. The dye-labeled lipid is a substrate for PI3 kinases, and the addition of the substrate to a PI3 kinase in the presence of ATP leads to the phosphorylation of the substrate. Addition of a sensor comprised of a fluorescent polymer co-located with metal ions on the surface of microspheres leads to binding of the phospholipid by sensor, which quenches the fluorescence of the fluorescent polymer in the sensor, and, optionally, re-emitting (sensitized emission) energy by the fluorescent quencher dye of the substrate. The assays may be monitored using any one of four different modes of readout: a) quenching of polymer emission (in one embodiment monitored at 490 nm); b) re-emission of energy by fluorescent dye (in one embodiment monitored at 574 nm); c) ratiometric quench (in one embodiment a ratio of 490 nm/574 nm) and d) ratiometric re-emission ( in one embodiment a ratio of 574 nm/490 nm). The ratiometric read-out modes allow for the elimination of most false positives and false negatives obtained in a compound screen.

According to one embodiment, a lipid which is labeled with a quencher is used to facilitate the detection of biological molecules such as inhibitors for lipid and polynucleotide kinases or phosphatases.

One embodiment makes it possible to detect kinase and phosphatase activity using lipids and/or nucleotides as substrates.

Nonlimiting examples of phosphorylatable lipid substrates include phosphatidic acid and its derivatives, phosphatidylethanolamine and its derivatives, phosphaidylcholine and its derivatives, phosphatidylserine and its derivatives, phosphatidylinositols and its derivatives, sphingomyelin and its derivatives, and cholesterol and its derivatives. Mixtures are possible.

Nonlimiting examples of phosphorylatable polynucleotide substrates include DNA, RNA, polynucleic acids, and the like, or a combination thereof. The application can also be used for identification and quantitation of non-enzymatic mediated phosphorylation of substrates including, but not limited to, inositol phosphates. Mixtures are possible.

The substrate may be bound to or labeled with the quencher by any suitable method, including NHS-ester couplings, carboxy coupling, sulfylhydryl couplings, or, for example, using secondary amine present on lysine.

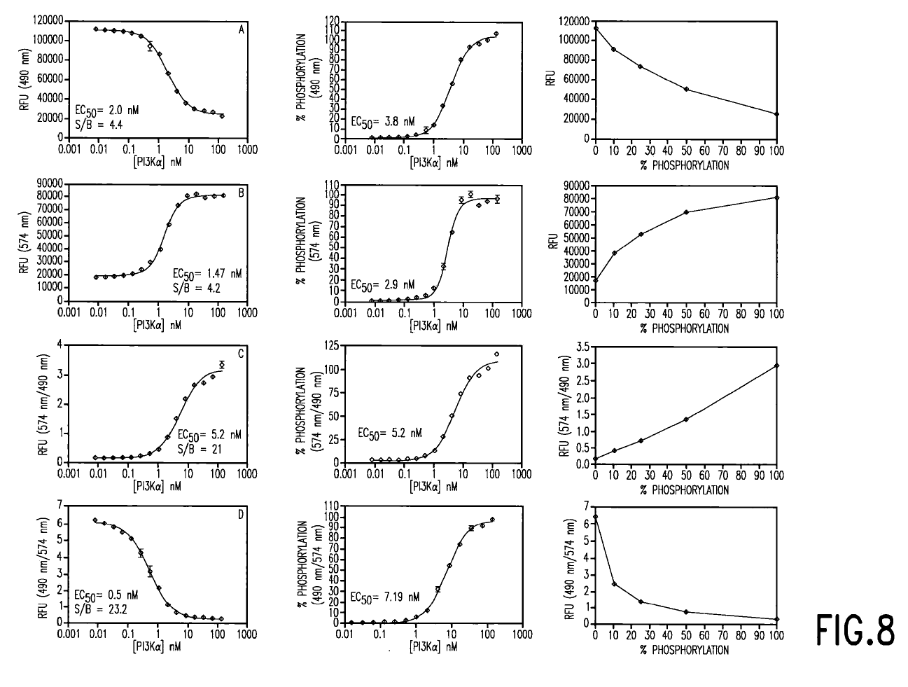

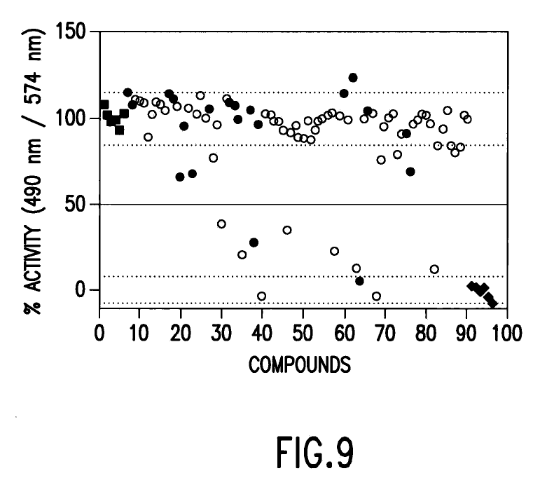

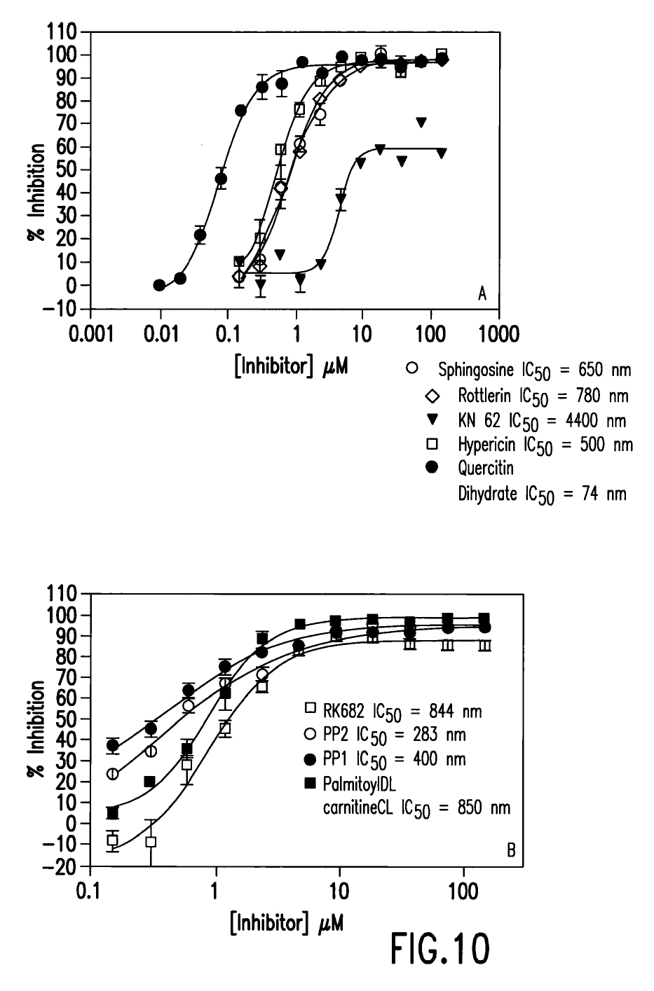

The assay is suitable for, inter alia, the detection of Class IA PI3K (PI3K α, β, β, and δ) activities. In one embodiment, BODIPY®TMR-labeled phosphatidylinositols are used as substrates. In one embodiment, the assay can be monitored as fluorescent polymer quench (FIG. 8A); as fluorescent dye re-emission (FIG. 8B); as ratiometric re-emission (FIG. 8C) and as ratiometric quench (FIG. 8D). The ratiometric re-emission method allows product conversion to be directly correlated to fluorescence signal, thus allowing the researcher to determine kinetics and optimal reaction conditions without the need for calibrator curves and backcalculations (FIG. 8C). The ratiometric quench method results in highly sensitive assays that allow for as little as 5% conversions to be detected. This method delivers high robustness (Z′ Factor) and a large signal window compared to single wavelength method and reduces or completely eliminates interference from autofluorescent and precipitating compounds. When applied to an 84 compound screen, the known inhibitors wortmannin, quercetin dihydrate and LY-294002 were correctly identified. No false positives were obtained (FIG. 9). FIG. 10 shows inhibition curves and IC₅₀ values of strong inhibitors that were identified in a screen against PI3K δ.

In one embodiment, the assay is based on metal-ion mediated superquenching of the fluorescent polymer.

One embodiment is directed to an assay for monitoring phosphoinositide-3 (PI3) kinase activities and for the identification of inhibitors of such kinases. Another embodiment is directed to pharmaceutical compositions comprising inhibitors of such kinases that can be used in the manufacture of medications to diseases that are responsive to inhibition of said kinases.

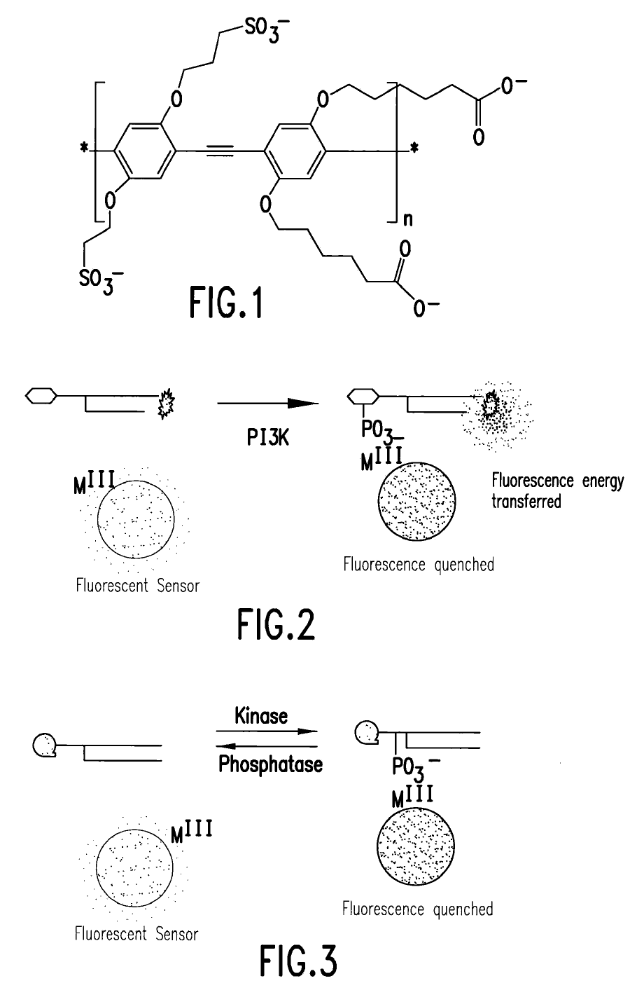

In one embodiment, the fluorescent polymer complexes with a metal species and contains a plurality of fluorescent moieties. In one embodiment, the fluorescent polymer is a conjugated (e.g., conducting or semiconducting) polymers such as poly(phenyleneethynylene) (PPE) (FIG. 1) and its soluble derivatives are known to exhibit photoluminescence with high quantum efficiency. Conjugated polymers have been demonstrated with emission colors that span the full range of the visible spectrum. Conjugated polymers and oligomers are polyunsaturated compounds in which all backbone atoms are sp- or sp2-hybridized. The chemical structures of these materials are represented by alternating single and double bonds, or alternating single and triple bonds, or alternating double and triple bonds. The electrons that constitute the double or triple bonds are delocalized over the entire molecule. Conjugated polymers and oligomers are composed of long chains of atoms with repeating structural units and delocalized electronic structure, and can be regarded as intrinsic semiconductors (and can be organic conductors). They may be made up completely of carbon atoms along their backbone, or contain other atoms such as nitrogen and sulfur for example.

By functionalizing the conjugated backbone of the polymers with suitable side chains, these polymers can be made soluble in common organic solvents and/or in water. The use of fluorescence superquenching (sometimes called “amplified superquenching”) in assays for kinase enzyme activity is advantageous in that the luminescence of a semiconducting polymer in aqueous solution can be quenched by using extremely low concentrations of an energy or electron acceptor (also called “quencher”). The superquenching effect is characterized by the quenching of more than one fluorescent chromophore of the polymer by one quencher. The mechanism for the quenching is believed to be ultrafast photoinduced electron or energy transfer. In the case of electron transfer, the excited electron transfers to a nearby acceptor within a few hundred femtoseconds, more than four orders of magnitude faster than the luminescence decay time. In the case of energy transfer, the polymer transfers the absorbed energy to the quencher. In either case, the quencher is excited while the polymer relaxes from its excited state. The quencher, if capable of emitting fluorescence in its own right, can re-emit fluorescence. The photoinduced electron or energy transfer step is exponentially sensitive to the distance separating the polymer chain from the quencher. If the quencher is removed from the vicinity of the polymer chain by ˜10 nm, the transfer rate will be so slow that the radiative recombination channel (luminescence) will again dominate (the quencher loses its ability to quench polymer fluorescence when separated by ˜10 nm).

Conjugated polymers in the poly(phenyleneethynylene) (PPE) family can be prepared with a variety of functional groups appended to the aromatic rings. Among the pendant anionic groups that have been used are those shown schematically in FIG. 1 which shows the molecular structure of a sulfo poly p-phenyleneethynylene (PPE-Di-COOH) conjugated polymer. Polymers with either of these functional groups associate with cationic microspheres in water to form stable polymer coatings. The resulting coated microspheres exhibit strong fluorescence. The overall charge on the polymer-coated microspheres can be tuned by the degree of polymer loading and by varying the structure of the polymer. The polymer coated microspheres can associate with metal cations and anions, and the loading of metal ions may depend on the loading level of the polymer on the microsphere. Certain metal ions such as Fe³⁺ and Cu²⁺ can quench the polymer fluorescence while others such as Ga³⁺ and related gallium species do not. Different metals may be suitably selected according to the desired application.

Upon addition of a phosphorylated peptide containing a quencher (e.g., a dye) which serves as a good energy transfer acceptor for the polymer donor (FIG. 3), little or no quenching of the fluorescence of aqueous suspensions of polymer-coated microspheres was found. After the polymer-coated microspheres were “charged” by addition of aqueous gallium chloride, however, addition of the same phosphorylated peptide to suspensions resulted in a pronounced quenching of the polymer fluorescence. Peptides containing only a phosphorylated residue or only the quenching dye produce little effect on the polymer fluorescence under the same conditions. Association of the phosphate on the peptide with the Ga³⁺ brings the quencher into close proximity with the polymer and mediates the fluorescence quenching.

The specific association of a phosphorylated biomolecule with the metal ion charged polymer is exploited to form the basis of a number of assays. FIG. 3 shows schematically the construction of one sensor based on metal ion mediated superquenching and an example for a kinase assay. In particular, FIG. 3 shows the phosphorylation or dephosphorylation of quencher labeled lipid substrates by target enzymes detected by the addition of a sensor comprising a fluorescent polymer and a metal. The lipid products are labeled with a quencher and brought to the surface of the polymer by virtue of specific phosphate binding to the metal ion (e.g., Ga³⁺). The resulting quench of polymer fluorescence is concomitant with phosphorylation or dephosphorylation.

Kinase/phosphatase assays are developed, in one embodiment, using lipid substrates. The present application makes it possible to enhance sensitivity in the measurement of enzymatic activity by amplifying the fluorescence signal using superquenching. In one embodiment, the sensor platform comprises a modified anionic polyelectrolyte poly(phenyleneethynylene), PPE derivative (see FIG. 1) which can be immobilized by adsorption on positively charged microspheres. This polymer exhibits photoluminescence with high quantum efficiency and has been used for detection of protease, kinase and phosphatase activities. A kinase/phosphatase assay is provided using fluorescent polymer superquenching for biodetection as shown in FIG. 3. Di- or trivalent metal ions can strongly associate with anionic conjugated polymers such as MPS-PPV and anionic PPE in solution, resulting in modification and/or quenching of polymer fluorescence. Since the overall charge on a polymer-microsphere ensemble can be tuned, these ensembles can be constructed to afford a platform whereby metal ions could be associated with the polymer without strongly quenching the polymer fluorescence and yet retaining the ability to complex with specific ligands. The approach is similar to that used in immobilized metal ion affinity chromatography (IMAC) whereby metal ions can specifically trap phosphorylated compounds by coordination with the phosphate oxygen at low pH.

Ga³⁺ can associate with microsphere-PPE ensembles without quenching the polymer emission. The PPE-associated Ga³⁺ can associate with phosphorylated lipids such that when the lipid contains a quencher (e.g., a dye such as rhodamine), metal ion mediated polymer superquenching occurs. This approach provides a basis for a sensitive and selective kinase/phosphatase assay as illustrated in FIG. 3. In the case of the fluorescence quench (turn off) kinase assay, the quench of polymer fluorescence is linear with enzyme activity. The assays are instantaneous, “mix and read” and require no wash steps or complex sample preparation.

Example 1 shows robust assays for phosphoinositol Class 1A (PI3K α, β, and δ) enzyme activities using phosphatidylinositol (PtdIns) as a substrate. The assays routinely deliver Z′ values of >0.8. In the example shown below the kinase assay functions as a fluorescence “turn off” assay. Since, for substrates such as those described, the quencher may exhibit sensitized fluorescence as a consequence of the quenching of polymer fluorescence, the assays can be used as either turn on or turn off, depending on the wavelength monitored. Additionally, monitoring simultaneously the fluorescence of the polymer and the quencher provides for a sensitive ratiometric assay. Additionally, detection could be carried out by monitoring fluorescence polarization in the quencher of the lipid substrate. For protein kinase and phosphatase assays using lipids as substrates which are based on metal ion mediated superquenching, both end point and kinetic assays may be carried out.

Novel assays have been developed using fluorescence superquenching for the measurement of phosphoinositol kinase (PI3K) enzyme activity. PI3K enzymes cause phosphorylation of a suitable lipid substrate. Measurement of this phosphorylation event is a direct measure of the kinase enzyme activity. A technique called immobilized metal ion affinity chromatography (IMAC) exists where metal ions are used to specifically trap phosphorylated molecules by coordination with the phosphate group at low pH. One embodiment includes the creation of a novel sensor comprising a fluorescent conjugated polymer complexed to metal ions wherein such complexation shows weak or no quenching of polymer fluorescence. One example of such a bioconjugate is utilized as the sensing element either on its own or in combination with a quencher dye, which is bound or complexed to the lipid substrate. The assay for kinase activity may be performed by mixing together the kinase with the lipid substrate. Phosphorylation of the lipid occurs. The product phospholipid associates with the sensor through metal-ion phosphate binding. This association facilitates close approach of the quencher to the polymer, whereby fluorescence of the polymer is quenched or reduced. The quencher can be either a non-fluorescent or a fluorescent molecule; in case of the latter, re-emission of fluorescence may occur. One embodiment includes assays of either type where quencher re-emission may or may not occur. In the former case, one embodiment includes the use of ratiometric measurement of fluorescence where the measured polymer emission and the measured quencher emission from the same sample are converted mathematically to a ratio or other equation.

A variety of fluorescent species have been shown to provide fluorescence superquenching in the presence of electron and energy transfer quenchers. The list of fluorescent species includes but is not limited to conjugated polyelectrolytes, charged conjugated polymers, neutral conjugated polymers, virtual polymers of the type exemplified by cyanine dye functionalized polylysines, conjugated polymer blends, and conjugated oligomers. The fluorescent species may or may not have functional groups capable of binding metal species. Any of these fluorescent species can be utilized either in solution or affixed to a solid support or both. Such support includes but is not limited to latex microspheres or beads, silica microspheres or beads, glass slides, nanoparticles, microtiter plates and colloids. The support may or may not have surfaces functionalized with organic or inorganic functional groups. Examples of organic functional groups include but are not limited to amine, amidine, quarternary amine, carboxylic acid, sulfonate and biotin. Examples of inorganic functional groups include but are not limited to transition metal ions such as iron and copper, alkali metal ions such as sodium and/or potassium ions, metal oxides, and metal nitrates. Mixtures of functional groups are possible. The fluorescent species may be affixed to the solid support by physical adsorption, coulombic attraction, covalent linkage, and the like.

One embodiment includes the assembly of a sensor comprising the fluorescent species either in solution or on solid support complexed to a metal species capable of binding phosphomolecules either weakly or strongly. The metal species comprises a metal in a form that includes but is not limited to an ion, elemental metal, oxide, nitrate, carboxylate, phosphate, and/or an organometallic species. Examples of metals that can be used in one embodiment include but are not limited to gallium, iron, copper, zinc, aluminum, nickel, cobalt, scandium, lutetium, cerium, lanthanum, thallium, thorium, ytterbium, or any combination thereof. On the solid support, the assembly of the sensor may occur either through complexation of the metal species by the polymer or by the solid support itself, and the assembly may occur in any sequence such as polymer on solid support followed by metal species, or metal species complexed to solid support followed by the polymer. The term “complex” refers to any metal-ligand association, for example, metal-ligand binding, adsorption, covalent linkage, ionic binding, Van der Waals interactions, physisorption, chemisorption, or electrodeposition.

One embodiment includes an assay for phosphoinositol-3 kinases wherein the enzyme of interest is detected and its activity measured by monitoring the change in fluorescence of the sensor when the said sensor is exposed to a mixture of the enzyme and its substrate. The enzyme may be either a recombinant species or wild-type. The enzyme may be one of any class of PI3 kinases or a sphingosine kinase or other lipid kinases. The sample containing the enzyme of interest is in the form of, but is not limited to, a purified sample or a cell lysate or a tissue extract.

The substrate is in the form of a lipid that may or may not contain one or more phospho groups and is bound or complexed to a quencher. The phospho group or phosphate refers to the chemical moiety represented by the formula “PO₄” or “OPO₃” in either the acid, salt, or ionic form.

The quencher on the substrate may be any electron transfer or energy transfer dye. Nonlimiting examples include a fluorescent or non-fluorescent dye, a fluorescent protein or a metal complex. In one embodiment, the quencher is capable of quenching polymer fluorescence by energy or electron transfer. In another embodiment, the quencher may or may not re-emit fluorescence. Nonlimiting examples of fluorescent or non-fluorescent dyes include BODIPY TMR, BODIPY TX, AlexaFluor 594, Texas Red, dinotrophenol (DNP), CY3, CY5, and fluorescin. Nonlimiting examples of fluorescent proteins include phycoerythrin and allophycocyanin. Nonlimiting examples of metal complex include iron-iminodiacetic acid and iron-nitroloacetic acid. In one embodiment, the binding or complexing refers to covalent linkage or avidin-biotin complex where the lipid is bound to either avidin or biotin, and the quencher is consequently bound to the respective biotin or avidin. In one embodiment, the substrate is biotinylated streptavidin. The quencher-labeled substrate may, in turn, be bound to a solid support.

In one embodiment, the assay is performed by mixing together the substrate and enzyme sample first, incubating for a finite time period, then contacting the sensor with the mixture; or by mixing together the substrate, enzyme sample, and sensor, incubating for a finite time period, before reading fluorescence. Thus, the assay may be performed in end-point or in real-time format.

Kinetic or real-time assays allow one to observe the mode of action of inhibitors and activator of enzyme activity in “real time”. Several inhibitor profiles are contemplated herein.

One type of inhibitor profile includes irreversible inhibitors with high or low binding affinity. Many ATP binding site inhibitors are ATP-competitive inhibitors and bind to the ATP binding pocket reversibly. Their inhibitory effect is then a function of their increased binding affinity compared to endogenous ATP. If an inhibitor however has a lower association constant than unmodified ATP but binds irreversibly, an inhibitor curve will initially appear as uninhibited, and reverse at a time point when an amount of compound has irreversibly bound in concentrations sufficient to affect the enzyme. Although such inhibitors will appear as “hits” in an endpoint measurement a kinetic profile will allow the researcher to have immediate knowledge of its action.

Another type of inhibitor profile includes inhibitors which act on sites of the enzyme other than ATP-binding pocket. Inhibitors may bind to sites of an enzyme which affects substrate binding and/or locks the enzyme into a conformation that inactivates it. Such inhibitors are likely to demonstrate different kinetics than ATP competitive inhibitors and can easily be identified by monitoring in real time.

Another type of inhibitor profile utilizes the mode of action of enhancers. Enhancers may cause a very steep increase in activity (compared to control) within several minutes of reaction and then flatten out over time to an activity equal to that of the control. Thus, endpoint measurement could miss these enhancers. For example, Trypohostin 35, Trypohostin 25 and GW 5074 are enhancers of PI3 alpha, beta, and delta, respectively, in real time, and result in a >50% increase of activity within 5 minutes of reaction compared to ˜20% when monitored in endpoint mode. Enhancers may not be of therapeutic value for PI3Ks (since their pathological effect is exerted mainly by over-activity to begin with) but knowledge of chemical structure of enhancers may facilitate identification and/or confirmation of environmental pathogens, which may cause disease.

Other advantages of kinetic monitoring include time saving and cost saving in assay development. For example, the reaction can be terminated after several minutes if no reaction is monitored and started over. Reactions conditions can be optimized as the reaction occurs, for example, through addition of more ATP, substrate etc. The substrate, ATP and enzyme progress curves can be monitored with one reaction mixture only as opposed to several that have to be performed in endpoint time courses, thus making assay development and optimization easier.

The assay mixtures herein may be monitored on any spectrophotometer capable of exciting samples and measuring fluorescence over the suitable wavelength range, such as UV, UV-Vis, or visible wavelength range including microtiter plate readers such as Molecular Devices' Spectramax Gemini series. Such methods usually measure percent absorption or transmission of specific wavelengths or wavelength regions of light in a bulk volume of sample. When the sensor comprises a solid support, the assay may also be monitored by measuring fluorescence per particle in a flow cytometer.

The assay mixture may be monitored either by measuring change in polymer fluorescence or change in quencher fluorescence or both. When measuring both, the two measurements may be used to interpret results independently or combined in a mathematical equation such as a ratio to interpret results. FIG. 2 shows schematically the construction of one sensor based on metal ion mediated superquenching and an example for a kinase assay.

In one embodiment, the assays are instantaneous, “mix and read” and require no wash steps or complex sample preparation. High tolerance of the sensor to adenosine triphosphate (ATP), the ability to miniaturize the assay to volumes compatible with high-throughput and ultra high-throughput screening (HTS and uHTS, respectively) and the high robustness of the assay (high Z′ Factor), make this platform attractive for rapid screening of compound libraries. In addition, the ability to read each sample at two wavelengths (when the quencher is capable of re-emission of fluorescence) allows for elimination of interference from colored and fluorescent library compounds.

In one embodiment, methods of detecting kinase and phosphatase enzyme activity employ metal ion mediated superquenching. Quencher dye labeled lipids and/or polynucleotides are used as substrates to facilitate screening of inhibitors for lipid and polynucleotide kinases or phosphatases. The methods herein are also suitable for the identification and quantitation of non-enzymatic mediated phosphorylation of substrates such as inositol phosphates. Reagents and kits for detecting lipid and polynucleotide kinase and phosphatase enzyme activity are also contemplated.

The fluorescence can be easily measured according to known spectrophotometric methods for measuring UV, visible, and UV-vis radiation, and the like. Similarly, the fluorescence of the compounds and compositions herein may be excited according to known methods.

The wavelengths may suitably range from 200-800 nm for excitation and from 200-800 nm for emission. In one embodiment, the wavelengths range from 300-700 nm for excitation and 400-800 nm for emission. In another embodiment, the wavelengths range from 400-600 nm for excitation and from 490-700 nm for emission. These ranges include all values and subranges therebetween, including 200, 300, 400, 410, 420, 430, 440, 450, 460, 470, 480, 490, 500, 510, 520, 530, 540, 550, 560, 570, 580, 590, 600, 610, 620, 630, 640, 650, 66, 670, 680, 690, 700, and 800 nm.

One embodiment relates to pharmaceutical compositions comprising one or more active ingredients identified by the assay and/or pharmaceutically acceptable salts thereof and one or more a pharmaceutically acceptable carrier, excipient, adjuvant and/or diluent. In one embodiment, the active ingredient is suitable in the prevention and/or treatment of a disease or condition responsive to inhibitors of PI3K enzymes.

One embodiment relates to a method for inhibiting or reducing the release of PI3K enzymes and/or inhibiting or reducing the activity of PI3K enzymes by administering the PI3K inhibitor to a subject. In one embodiment, the subject is known to be in need of such inhibition or reduction.

One embodiment includes a method of administering the active ingredient derived from the assay to a human subject. In another embodiment, the method includes administering the active ingredient to a human afflicted with a disease or condition which is responsive to an inhibitor of PI3K enzymes or in need of inhibiting or reducing the release of PI3K enzymes, and/or in need of inhibiting or reducing the activity of PI3K enzymes.

Other embodiments relate to methods of making and using the active ingredient identified by the assay.

The active ingredient, salt thereof, prodrug thereof, or a combination thereof can be administered in combination with one or more substantially nontoxic pharmaceutically acceptable carriers, excipients, adjuvants or diluents. The compositions may be prepared in any conventional solid or liquid carrier or diluent and optionally any conventional pharmaceutically-made adjuvant at suitable dosage level in a known way. The preferred preparations are in administrable form which is suitable for oral application. These administrable forms, for example, include pills, tablets, film tablets, coated tablets, capsules, powders and deposits.

Forms other than orally administrable forms are also possible. In one embodiment, active compounds and/or pharmaceutical preparations containing said compounds may be administered by any appropriate means, including but not limited to injection (intravenous, intraperitoneal, intramuscular, subcutaneous) by absorption through epithelial or mucocutaneous linings (oral mucosa, rectal and vaginal epithelial linings, nasopharyngial mucosa, intestinal mucosa); orally, rectally, transdermally, topically, intradermally, intragastrally, intracutanly, intravaginally, intravasally, intranasally, intrabuccally, percutaneously, sublingually, or any other means available within the pharmaceutical arts.

The pharmaceutically acceptable carrier may be suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the salt may be combined with any oral nontoxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent by weight of the inventive compound, salt thereof, or a mixture of compound and salt, which range includes all values and subranges therebetween, including 10, 15, 20, 25, 30, 35, 40, 45, 50, 55, 60, 65, 70, 75, 80, 85, and 90% by weight.

Suitable binders include starch, gelatin, natural sugars, corn sweeteners, natural and synthetic gums such as acacia, sodium alginate, carboxymethyl-cellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compounds or compositions may be formulated in sustained release form to provide the rate controlled release of any one or more of the components or active ingredients to optimize the therapeutic effects, e.g., inhibition and/or reduction of PI3K enzyme and/or activity, and the like. Suitable dosage forms for sustained release include layered tablets containing layers of various disintegration rates or controlled release polymeric matrices impregnated with the active components and shaped in tablet form or capsules containing such impregnated or encapsulated porous polymeric matrices.

Liquid form preparations include solutions, suspensions and emulsions. Nonlimiting examples include water, ethanol, ethanolic, water-ethanol or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The compounds may also be deliverable transdermally. The transdermal compositions may take the form of creams, lotions, aerosols and/or emulsions and can be included in a transdermal patch of the matrix or reservoir type as are conventional in the art for this purpose.

The term capsule refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art.

Oral gels refer to the active ingredients dispersed or solubilized in a hydrophillic semi-solid matrix.

Powders for constitution refer to powder blends containing the active ingredients and suitable diluents which can be suspended in water or juices.

Suitable diluents are substances that usually make up the major portion of the composition or dosage form. Suitable diluents include sugars such as lactose, sucrose, mannitol and sorbitol, starches derived from wheat, corn rice and potato, and celluloses such as microcrystalline cellulose. The amount of diluent in the composition can range from about 5 to about 95% by weight of the total composition, preferably from about 25 to about 75%, more preferably from about 30 to about 60% by weight.

The term disintegrants refers to materials added to the composition to help it break apart (disintegrate) and release the medicaments. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. The amount of disintegrant in the composition can range from about 2 to about 20% by weight of the composition, more preferably from about 5 to about 10% by weight.

Binders characterize substances that bind or “glue” powders together and make them cohesive by forming granules, thus serving as the “adhesive” in the formulation. Binders add cohesive strength already available in the diluent or bulking agent. Suitable binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropyl-methylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d,1-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidents are materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents are excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Other techniques for the formulation and administration of the active ingredients may be found in “Remington's Pharmaceutical Sciences” Mack Publishing Co., Easton Pa., the entire contents of which are hereby incorporated by reference. A suitable composition comprising at least one compound may be a solution of the compound in a suitable liquid pharmaceutical carrier or any other formulation such as tablets, pills, film tablets, coated tablets, dragees, capsules, powders and deposits, gels, syrups, slurries, suspensions, emulsions, and the like.

The term “treating” as used herein refers to reversing, alleviating, inhibiting the progress of, or preventing the disorder or condition to which the term applies, or one or more symptoms of the disorder or condition. The term “treatment” as used herein refers to the act of treating as the term is defined above.

Active ingredients may exist in any convenient liquid, solid, crystalline, semicrystalline, or amorphous form. Solid forms may be achieved via typical crystallization routes including vacuum crystallization or spray drying. Depending on the solublity desired, the amorphous form obtained by, e.g., spray-drying may be preferred. The spray drying may be carried out from aqueous, ethanolic, organic, or mixed aqueous ethanolic solutions of the salt or a mixture of the salt and the free base compound. The compound and/or salt may exist in a form comprising one or more waters of hydration.

The active ingredient may suitably be administered in the form of a salt of a pharmaceutically acceptable acid or base, as appropriate. In one embodiment, the pharmaceutically acceptable salt desirably satisfies one or more of the following physiochemical criteria: (1) good solubility; (2) good stability; (3) non-hydroscopicity; and (4) processability for oral, parenteral, or topical formulations, etc.

Suitable pharmaceutically administrable salts desirably have good aqueous solubility and good bioavailability. Usually a solubility of greater than 1 mg/mL at pH 1-7.5 is sought although higher solubilities are required to formulate injections. In addition, salts which provide solutions having a pH close to that of blood (7.4) are preferred because they are readily biocompatible and can easily be buffered to the required pH range without altering their solubility.

Polynucleotide Kinase Activity Monitored by Metal Ion Mediated Fluorescence Superquenching

PNK enzymes were first discovered in Escherichia coli infected with bacteriophage T4 and T2. Mammalian polynucleotide kinase (PNK) is a key component of both the base excision repair (BER) and non-homologous end-joining (NHEJ) DNA repair pathways. PNK acts as a 5′-kinase/3′-phosphatase to create 5′-phosphate/3′-hydroxyl termini, which are a necessary prerequisite for ligation during repair. PNK is recruited to repair complexes through interactions between its N-terminal FHA domain and phosphorylated components of either pathway. The kinase domain has a broad substrate binding pocket, which preferentially recognizes double-stranded substrates with recessed 5′-termini. In contrast, the phosphatase domain efficiently dephosphorylates single-stranded 3′-phospho termini as well as double-stranded substrates. PNK activity is linked to drug resistance in chemotherapies and radiation therapies by repairing therapy induced DNA damage, which would otherwise result in cell death. Identification of inhibitors to PNKs is therefore an attractive approach to preventing drug resistance and/or increasing sensitivity to therapies.

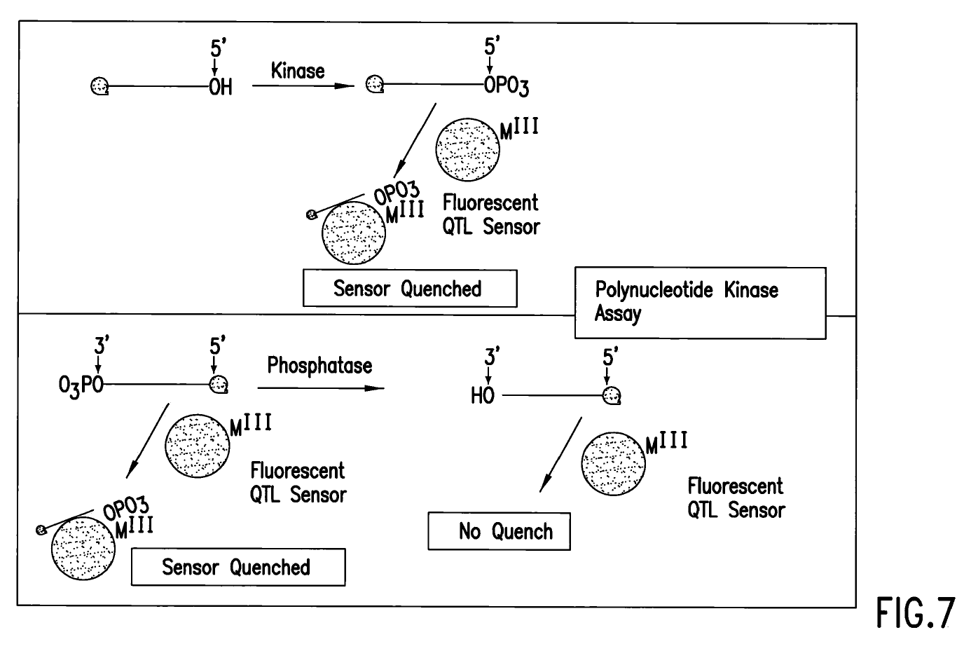

The use of fluorescent polymer superquenching based assays for the detection of PNK activity provides enhanced sensitivity and an easy approach to perform high-throughput screening of compound libraries. In one embodiment, quencher-labeled oligonucleotides are utilized having a 5′-OH group that can be phosphorylated by a PNK and/or a 3′-PO₃ group that can be dephosphorylated by the PNK. Upon phosphorylation of the 5′-OH group by PNK, the oligonucleotide is able to bind the sensor through metal-phosphate binding interactions. The phosphatase activity of PNK can be measured through a reverse process where the loss of the 3′-PO₃ group from the oligonucleotide prevents its binding to the sensor. An assay format of this type is illustrated in FIG. 7, which is a schematic illustrating an assay for polynucleotide kinase or phosphatase activity. The fluorescent polymer sensor-based assay platform can therefore be utilized in the development of an assay for polynucleotide kinase or phosphatase activity. The assay can be employed in a high-throughput screening format to detect inhibitors of enzyme activity.

### EXAMPLES

The following examples are not intended to be limiting.

Ratiometric Assays for Phosphoinositide 3-Kinase (PI3K) Family of Enzymes

For detection of PI3K activity rhodamine-labeled phosphoinositol and the calibrator were obtained from Echelon (Salt Lake City, Utah). Recombinant PI3 kinases α, β, and γ were from Upstate (Lake Placid, N.Y.). The inhibitor wortmannin for PI3K activity was purchased from Upstate and inhibitor LY-294002 was from Calbiochem (San Diego, Calif.). Polystyrene amine functionalized beads were obtained from Interfacial Dynamics. The polymer (1) was chosen as a sensor for kinase/phosphatase assays based on our recent discovery that di- or trivalent metal ions can strongly associate with anionic PPE in solution. No quench of emission was observed when GaCl₃ in a concentration of 340 M was added to a microsphere solution. It was observed that rhodamine-labeled phospho-lipids provided a strong quench of polymer fluorescence whereas little modulation of fluorescence was observed when non-phosphorylated rhodamine-labeled lipids were used.

FIG. 2 shows a schematic representation of PI3 kinase assay. The forked lines represent PtdIns labeled with a quencher (starburst). Light circles represent fluorescence unquenched sensor. Following metal-ion mediated association of phosphorylated PtdIns to the sensor fluorescence can be monitored as quench in a “turn off” assay (darker circle) or as a “turn on” assay via transferred energy emission from sensor to fluorescent dye (darkened hue on right of figure near rightmost starburst).

As depicted in FIG. 3, assays have been developed using quencher-labeled, synthetic lipids as substrates. Upon phosphorylation of the substrate, the lipid can associate with the sensor via the phosphate groups thereby quenching fluorescence.

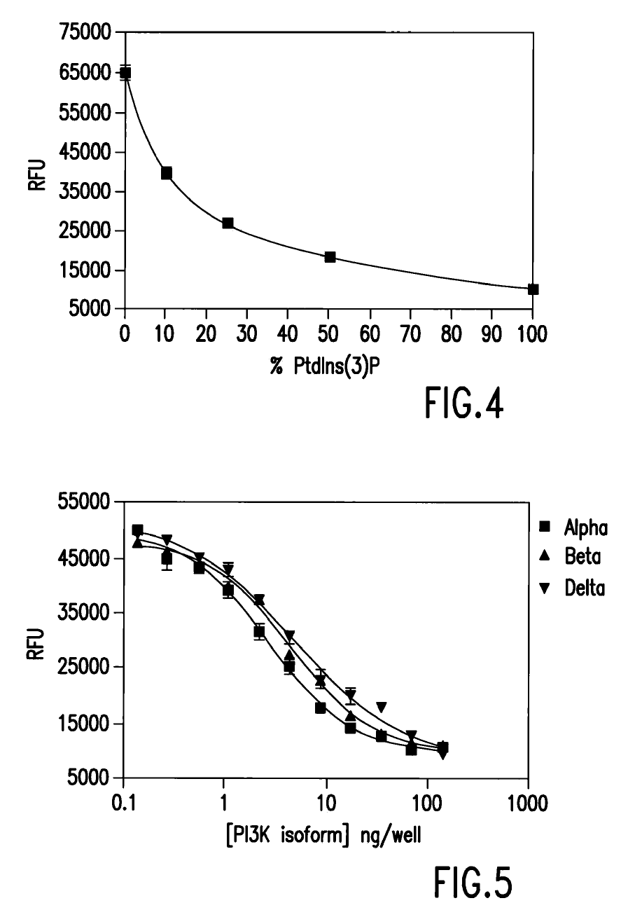

FIG. 4 shows a calibration curve in which phosphorylated and non-phosphorylated lipids were mixed in various ratios and combined with the sensor to detect the change in fluorescence signal as a function of phosphorylation. FIG. 4 shows calibration curves for phosphatdilyinositols.

FIG. 5 shows enzyme concentration curves. In particular, FIG. 5 shows an endpoint measurement of PI3 kinase activity in which an increase in polymer quench correlates with enzyme concentration.

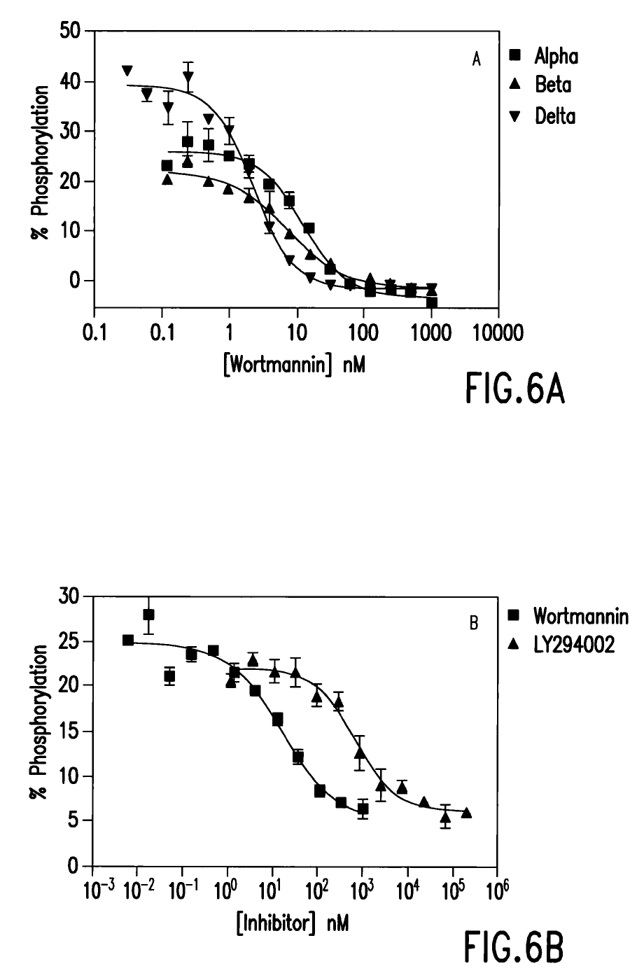

FIG. 6 shows the inhibition of PI3K α, β, and δ activity by wortmannin (FIG. 6A) and inhibition of PI3K α by LY-294002 (FIG. 6B). First, the sensitivity of the assay was tested by using a known inhibitor of PI3K activities, wortmannin (FIG. 6A). The IC₅₀ values obtained using 1 μM substrate in reactions with 200 μM ATP and 9 ng PI3K are 2.3 nM, 7.4 nM and 12.6 nM for PI3Kα, βand δ, respectively. The values are in agreement with published values (2-5 nM). An inhibitor of PI3Kα activity, LY-294002 was also tested (FIG. 6B) and delivered an IC₅₀ value of 0.6 μM, which is in close agreement to the published values of 0.5 μM -1.5 μM.

The statistical parameters that can be delivered with this assay were determined by evaluating known amount of phospholipids PtdIns in replicates of 12. This data is shown below in Tables 1 and 2.

TABLE 1
Statistics for PI3K α Kinase assays

| | Z′ | Signal/ | | | |
|---|---|---|---|---|---|
| Phosphorylation (%) | Values* | Noise | S/B | % CV | Signal Window |
| 0 | — | — | — | 4.5 | — |
| 21 | 0.54 | 9.1 | 1.9 | 5.8 | — |
| 48 | 0.64 | 11.6 | 3.0 | 10.8 | — |
| 100 | 0.81 | 18.3 | 5.9 | 5.5 | 22.5 |

*n = 12

TABLE 2
Statistics for PI3K β Kinase Assays

| | Z′ | | | | Signal |
|---|---|---|---|---|---|
| Phosphorylation (%) | Values* | Signal/Noise | S/B | % CV | Window |
| 0 | — | — | — | 2.7 | — |
| 22 | 0.63 | 11.4 | 2.0 | 6.8 | — |
| 55 | 0.79 | 19.7 | 3.5 | 8.3 | — |
| 95 | 0.86 | 28.2 | 5.4 | 5.6 | 37.5 |

*n = 12

The data shown in Tables 1 and 2 are excellent and show that this assay is suitable to determine as little as 20% conversion with Z factors of 0.54 and 0.63 and Z factors of >0.8 at 100% substrate conversion for PI3K α and PI3K β, respectively.

Assays for Identification and Confirmation of Inhibitors of Phosphoinositide 3-Kinases

For detection of PI3K activity, BODIPY®TMR-labeled Phosphatidylinositol and the phospholipid calibrator were purchased from Echelon (Salt Lake City, Utah). Recombinant PI3 kinases α, β and δ were purchased from Upstate (Lake Placid, N.Y.). The inhibitor library was purchased from Biomol (Plymouth Meeting, Pa.). The sensor was made using Polystyrene amidine functionalized microspheres obtained from Interfacial Dynamics (Eugene, Oreg.). For this example, a conjugated polymer 1 (FIG. 1) was chosen as the fluorescent species, gallium(III) chloride was used as the metal species and Polystyrene based amidine functionalized microsphere of diameter 0.45 μm were used as solid support in the sensor. The sensor ensemble was constructed in two steps. In the first step, physical adsorption of the conjugated polymer to the surface of the microspheres was performed, said physical adsorption achieved by mixing together the microsphere suspension and the polymer solution for a finite time period. The first step was followed by the second step wherein the polymer-microsphere ensemble was treated with gallium(III) chloride for a finite time period. This resulted in the binding of gallium to the polymer-microsphere ensemble as monitored by an assay for the amount of gallium in solution before and after treatment with the polymer-microsphere ensemble. Upon mixing the sensor with BODIPY®TMR labeled phospho-lipids, a strong quench of polymer fluorescence and re-emission from the BODIPY TMR dye was observed whereas little modulation of fluorescence was observed when non-phosphorylated BODIPY®TMR-labeled lipids were used. Increasing amounts of dye-labeled phospho-lipid provided increasing changes in polymer and quencher fluorescence, thus forming a strong basis for the determination of enzyme activity.

The principle was applied to assays for detection of Class I PI3K (PI3K α, β, γ and δ) activities using BODIPY®TMR-labeled phosphatidylinositols as substrates. FIG. 8 shows enzyme concentration curves for PI3K α in various read-out modes. Decreasing concentrations of PI3K α, were mixed with substrate to achieve a final concentration of 250 nM in assay buffer containing 250 μM ATP. After one hour incubation at room temperature, an equal volume of sensor (15 μL) was added. Calibrator curves were contructed simultaneously with the enzymatic reactions and product conversion determined. The identical experimental wells were monitored at emissions at 490 nm (A), 574 nm (B), 574 nm/490 nm (C) or 490 nm/574 nm (D) after excitation of samples at 450 nm. Data are presented as raw RFU (left panel), converted data (middle panel) and calibrator curves (right panel). The present inventors show that one enzyme reaction can be monitored as polymer fluorescence quench (FIG. 8A); as quencher dye re-emission (FIG. 8B); as ratiometric quench (FIG. 8C) and as ratiometric re-emission (FIG. 8D). The re-emission mode of ratiometric monitoring resulted in assays in which product conversion directly correlated to fluorescence signal, thus allowing determination of kinetics and optimal reaction conditions without the need for calibrator curves and backcalculations (FIG. 8C). The quench ratiometric mode resulted in highly sensitive assays that allowed for as little as 5% product formation to be detected. This mode delivered high robustness (Z′ Factor) and a large signal window compared to single wavelength mode and eliminated interference from autofluorescent and precipitating compounds. When applied to an 84 compound screen, the known inhibitors Wortmannin, Quercetin Dihydrate and LY-294002 were correctly identified. FIG. 9 shows compound screen against PI3K δ. Percent activities are plotted for each compound screened using the ratio of wavelengths at 490 nm/574 nm. Inhibition values were calculated relative to positive and negative controls wells and corrected for background (filled squares, filled diamonds). Empty circles represent compounds, and filled circles colored compounds. Dotted lines represent ±3 standard deviations for the mean RFU of control wells. No false positives were obtained (FIG. 9). FIG. 10 shows inhibition curves and IC₅₀ values of strong inhibitors that were identified in a screen against PI3Kδ. FIG. 10 shows determination of IC₅₀ values for PI3K δ positive hits: IC₅₀ values were obtained using inhibitor concentrations ranging from 150 μM to 146 nM in the presence or absence of enzyme. Quercetin Dihydrate concentrations ranged from 10 μM to 10 nM. Final concentrations of enzyme, substrate and ATP were 2.6 nM, 250 nM and 50 μM. The delta RFU between reactions in the presence and absence of enzyme were obtained and used to calculate % inhibition.

The entire contents of each of the following references is independently incorporated herein by reference for all purposes.

Chen, L. Et al, Proc. Natl. Acad. Sci. 1999, 96, 12287.

Chen, L. et al, Chem. Phys. Lett. 2000, 330, 27.

Chen, L. Et al, J. Am. Chem. Soc. 2000, 122, 9302.

Jones, R. M. et al, Langmuir 2000, 17, 2568.

Jones, R. M. et al, J. Am. Chem. Soc. 2001, 123, 6726.

Jones, R. M. et al, Proc. Natl. Acad. Sci. 2001, 98, 14769.

Kushon, S. A. Et al, Langmuir 2002, 18, 7245.

Lu, L. Et al, J. Am. Chem. Soc. 2002, 124, 483.

Kumaraswamy, S. et al, Proc. Natl. Acad. Sci. 2004, 101, 7511.

Xia, W. et al, Assay Drug Develop. Technol. 2004, 2, 183.

Rininsland, F. et al, Proc. Natl. Acad. Sci. 2004, 101, 15295.

Vanhaesebroeck, B. and Waterfield, D. Exp. Cell Res. 1999, 253, 239.

Stein, R. C. and Waterfield, D. M. Mol. Med. Today 2000, 6, 347.

Andersson, L. and Porath, J. Anal. Biochem. 1986, 154, 250.

Posewitz, M. C. and Tempst, P. Anal. Chem. 1999, 71, 2883.

Jinzhang, G. Et al. Rare Metals 2003, 22(1), 1.

Auger, K. R., Serunian, L. A., Soltoff, S. O., and Cantley, L. C. Cell 1989, 57, 167.

Richardson, C. C. Proc. Natl. Acad. Sci. USA 1965, 54, 158.

In addition, the entire contents of each of the following references is independently incorporated herein by reference for all purposes.

U.S. Provisional Application Ser. No. 60,662,783, filed Mar. 18, 2005.

U.S. Provisional Application Ser. No. 60/705,495, filed Aug. 5, 2005.

U.S. application Ser. No. 11/008,935, filed Dec. 13, 2004.

U.S. application Ser. No. 10/226,300, filed Aug. 23, 2002.

While the foregoing specification teaches the principles of the present invention, with examples provided for the purpose of illustration, it will be appreciated by one skilled in the art from reading this disclosure that various changes in form and detail can be made without departing from the true scope of the invention.

1. A method, comprising:
contacting,
an analyte;
a phosphorylatable or dephosphorylatable substrate bound to a quencher; and
a sensor comprising at least one fluorescent polymer complexed to one or more metal species;
to form a composition; and
determining the fluorescence of the composition, if any;
wherein when the substrate is phosphorylated, an association occurs between the substrate and the sensor such that fluorescent superquenching occurs between the fluorescent polymer and the quencher.

2. The method of claim 1, wherein the analyte is selected from the group consisting of a polynucleotide, lipid, kinase, phosphatase, enzyme, protein, peptide, and a combination thereof.

3. The method of claim 1, wherein the analyte is a polynucleotide.

4. The method of claim 1, wherein the analyte is a lipid.

5. The method of claim 1, wherein the analyte is a kinase.

6. The method of claim 1, wherein the analyte is a phosphatase.

7. The method of claim 1, wherein the substrate is specific to the analyte.

8. The method of claim 1, wherein the substrate is phosphorylated by the analyte.

9. The method of claim 1, wherein the substrate is dephosphorylated by the analyte.

10. The method of claim 1, wherein the fluorescent polymer and metal species form a salt.

11. The method of claim 1, wherein the fluorescence of the composition is indicative of the specificity of the substrate to the analyte.

12. The method of claim 1, further comprising determining the fluorescence of the fluorescent polymer, quencher, or both, in the absence of the analyte.

13. The method of claim 1, wherein determining the fluorescence further comprises determining a first fluorescence of the fluorescent polymer, if any, and determining a second fluorescence of the quencher that is different from the first fluorescence.

14. The method of claim 1, wherein determining the fluorescence further comprises determining a first fluorescence of the fluorescent polymer, if any, and determining a second fluorescence of the quencher that is different from the first fluorescence, and calculating a ratio of the first and second fluorescences.

15. The method of claim 1, wherein the sensor further comprises a solid support in contact with the fluorescent polymer, metal species, or both.

16. A method for detecting an inhibitor and/or activator of PI3 kinase or phosphatase activity, comprising:
contacting,
an analyte;
a phosphorylatable or dephosphorylatable substrate, specific to PI3 phosphatase or kinase, bound to a quencher; and
a sensor comprising at least one fluorescent polymer complexed to one or more metal species;
to form a composition; and
determining the fluorescence of the composition, if any;
wherein when the substrate is phosphorylated, an association occurs between the substrate and the sensor such that fluorescent superquenching occurs between the fluorescent polymer and the quencher.

17. The method of claim 16, wherein the analyte is an inhibitor and/or activator of PI3 kinase or phosphatase activity.

18. The method of claim 16, wherein determining the fluorescence further comprises determining a first fluorescence of the fluorescent polymer, if any, and determining a second fluorescence of the quencher that is different from the first fluorescence, and calculating a ratio of the first and second fluorescences.

19. An assay kit for probing an analyte, comprising:
a phosphorylatable or dephosphorylatable substrate bound to a quencher; and
a sensor comprising at least one fluorescent polymer complexed to one or more metal species;
wherein when the sensor and the substrate bound to the quencher are present in intimate admixture, and wherein when the substrate is phosphorylated, an association occurs between the substrate and the sensor such that fluorescent superquenching occurs between the fluorescent polymer and the quencher.

20. The kit of claim 19, further comprising a means for determining the fluorescence of the intimate admixture.
